(19) United States
(12) Patent Application Publication
(10) Pub. No.: US 2007/0196843 A1
(43) Pub. Date: Aug. 23, 2007

(54) Method for identification and monitoring of epigenetic modifications

(76) Inventors: Roland D. Green, Madison, WI (US); Hans T. Bjornsson, Baltimore, MD (US); Andrew P. Feinberg, Baltimore, MD (US)

Correspondence Address: QUARLES & BRADY LLP, 33 E. MAIN ST, SUITE 900, P.O BOX 2113, MADISON, WI, 53701-2113, US

(21) Appl. No.: 11/638,004

(22) Filed: Dec. 13, 2006

Related U.S. Application Data
(60) Provisional application No. 60/749,924, filed on Dec. 13, 2005.

(51) Int. Cl. C12Q 1/68; G06F 19/00

(52) U.S. Cl. 435/6; 702/20

(57) ABSTRACT

The present invention provides novel methods for identifying and monitoring epigenetic modifications, such as imprinted genes, using microarray based technology. Specifically, the invention detects imprinted genes by the presence of overlapping closed and open chromatin markers. The invention also discloses a method for detecting the loss of imprinting on a genome-wide scale, which is indicative of a variety of medical conditions. Diagnostic assays and chromatin structure markers for identifying gene imprinting and loss thereof are also disclosed.

### CROSS-REFERENCE TO RELATED APPLICATIONS

This application claims the benefit of U.S. Provisional Application No. 60/749,924 filed Dec. 13, 2005 which is incorporated herein by reference in its entirety.

### STATEMENT REGARDING FEDERALLY SPONSORED RESEARCH OR DEVELOPMENT

Not Applicable

### BACKGROUND OF THE INVENTION

According to the traditional laws of Mendelian genetics, people inherit two copies of their genes—one from their mother and one from their father. Usually, both copies of each gene are active, or “turned on,” in cells. In some cases, however, only one of the two copies is normally turned on. Which copy is active depends on the parent of origin. Some genes are active only when they are inherited from a person's father and others are active only when inherited from a person's mother. This phenomenon is known as “genomic imprinting.” In genes that undergo genomic imprinting, the parent of origin is often marked (i.e., through the process of methylation) on the gene during the formation of egg and sperm cells. This marking identifies which copy of a gene was inherited from the mother and which was inherited from the father.

Evidence for genomic imprinting originated from study of the whole genome, then progressed to studies of individual chromosomes and regions, and resulted in the identification of specific imprinted genes. Specifically, whole genome evidence for imprinting came from trisomic mouse and human embryos, which have markedly different phenotypes depending on the parental origin of the excess genome. Similarly, both mouse and human chromosomes that undergo uniparental disomy or UPD (duplication of one parental copy and loss of the other parental copy) often show characteristic phenotype alterations in the offspring. These can include overgrowth in the case of paternal UPD for some chromosomal regions, and growth retardation in the case of maternal UPD of the same chromosomal regions. There is a strong relationship between imprinted genes and both pre- and postnatal growth. Imprinting is also thought to underlie some quantitative trait loci relating to cell growth. As such imprinting may be a potential barrier to stem cell transplantation.

Chromosomes that likely harbor imprinted genes include at least 1, 2, 5, 6, 7, 11, 14, 15, 16, 18, 19, 20, and X. Specific human disease that involve imprinted genes include: Prader-Willi syndrome and Angelman syndrome, which result in short stature, mental retardation, and behavioral disorders; Beckwith-Wiedemann Syndrome (BWS), which causes prenatal overgrowth and predisposition to Wilms tumor, hepatoblastoma, and neuroblastoma; and pseudohypoparathyroidism type IA, which results in osteodystrophy and gonadal dysfunction. Furthermore, aneuploidy itself is a significant risk factor for malignancy, suggesting a role for subtle changes in gene dosage in cancer predisposition.

In addition, many complex traits show preferential transmission from a specific parent. For this reason, several common diseases are thought to involve an imprinted gene as one of the contributing genes, and these diseases include schizophrenia, bipolar affective disorder, autism, diabetes, and cancer. A role for imprinting and cancer was suggested indirectly twenty years ago by the observation that hydatidiform moles and ovarian teratomas arise from androgenetic (46 chromosomes, all of paternal origin) and parthenogenetic (all maternal chromosomes) embryos, respectively. Thus, even if the normal number of chromosomes is present, neoplasia forms an imbalance between maternal and paternal chromosomes.

Furthermore, an excess of the paternal genome leads to a more aggressive tumor than an excess of the maternal genome, suggesting that maternal and paternal chromosomes have differing effects on growth homeostasis. Alternatively, loss of imprinting (LOI) is the abnormal activation of the normally silent allele of an imprinted growth promoting gene such as IGF2, and/or abnormal silencing of the normally silent allele of an imprinted tumor suppressor gene such as p57/KIP2. LOI need not require complete erasure of an imprinting mark. Also, we and other researchers have found that LOI of IGF2 is one of the most common genetic alterations in cancer. These include embryonal tumors of childhood (epatoblastoma, rhabodomyosarcoma, and Ewing's sarcoma) and major adult malignancies (uterine, cervical, esophageal, prostate, lung, and germ cell tumors).

General approaches used to identify imprinted genes have included subtraction hybridization using maternally or paternally disomic mouse embryos, and 2-D gel electrophoresis of doubly-digested DNA from mice. However, these methods have shown comparatively low sensitivity and specificity. A major limitation to identifying imprinted genes has been that imprinting in mouse and humans is not identical. Furthermore, many of the approaches that have been used for the mouse are not ethically suitable for the human (e.g., involve embryo experimentation). An alternative slower approach has been to search for imprinted genes near other imprinted genes since, it has been found that imprinted genes tend to cluster together in the same chromosomal regions. However, this approach is limited to genes in proximity to known imprinted genes, which are relatively few because only a small percentage of all human genes undergo genomic imprinting.

Despite the great public health importance of imprinted genes, and the large amount of effort that has been expended by many laboratories, relatively little progress has been made in developing new methods for identifying imprinted genes. Thus, there remains a need for a diagnostic method to identify imprinted genes through screening and detection of loss of imprinting, which can indicate the presence or the risk of developing a disease.

### BRIEF SUMMARY OF THE INVENTION

The present invention is summarized as a novel method for rapidly identifying and monitoring epigenetic modifications, such as imprinted genes on a genome-wide scale. The method is based on the idea that one copy of an imprinted gene is active on the first chromosome and the other copy is inactive on the second chromosome. The promoter of the active gene is hypothesized to reside in an open chromatin region of the first chromosome and the promoter of the inactive copy of the imprinted gene resides on a closed (condensed) chromatin region of the second chromosome.

Applicants have been able to use microarray based methods, such as chromatin immunoprecipitation microarray, ChIP Chip™ technology to establish a map of genomic regions having at least one set of overlapping open and closed chromatin structure markers to identify imprinted genes. Alternatively, loss of imprinting can be identified using other array or PCR based techniques by observing the presence of only open chromatin structure markers in genomic regions where overlapping open and closed chromatin markers are generally observed. Thus, the imprinted genes and the loss of such imprinting, which may be associated with disease can be identified as described in the Examples below.

In a broad aspect, the invention encompasses the identification and assay of any epigenetic modification caused by allele specific expression, which includes but is not limited to imprinted genes.

In another aspect, the invention provides a method of identifying at least one imprinted gene in a subject characterized by the presence of at least one set of overlapping open and closed chromatin markers on a map established by analyzing chromatin structure of the subject's genome by the ChIP Chip™ technique. Such a method enables quick screening of a genome to identify active and inactive genes in a particular tissue or cell.

In another aspect, the invention provides a method of detecting the loss of gene imprinting in a subject using ChIP Chip™ technique, wherein the loss of imprinting is characterized by the loss of the closed chromatin markers, which is indicative of disease.

In this aspect, the invention provides a diagnostic assay or test for identifying loss of imprinting, which is useful in assessing a subject's predisposition to a disease, such as cancer or other associated diseases.

In a related aspect, the invention provides a method for assessing the risk of contracting a disease in a subject as a result of loss of imprinting.

In a related aspect, the invention provides a method for detecting the presence of a disease in a subject as a result of loss of imprinting.

In a related aspect, the invention provides a method for determining if a preimplantation embryo has an increased risk of developing a disease due to loss of imprinting.

In another aspect, the invention provides a method of establishing an imprinting map used to search for a disease.

Another aspect of the invention provides markers of chromatin structure for identifying gene imprinting and loss of such imprinting.

In this aspect, the markers include histone modifications and the presence of RNA polymerase II. The histone modifications include, for example: 1) acetylated lysine 9 of histone 3 (AcK9H3); 2) tri-methyl lysine 9 of histone 3 (3MeK9H3); and 3) di-methyl lysine 9 of histone 3 (2MeK9H3).

A related aspect of the invention is the ability to produce a relatively small, high quality set of imprinted genes for diagnostic use.

Other objects advantages and features of the present invention will become apparent from the following specification.

### BRIEF DESCRIPTION OF THE SEVERAL VIEWS OF THE DRAWINGS

FIG. 1 shows a schematic flowchart of the steps associated with performing ChIP Chip™ experiments in accordance with the invention.

FIGS. 2A and B are plots from ChIP Chip™ experiments showing overlapping regions of both open and closed chromatin markers. (A) The first track shows the AcK9H3 marker, an open chromatin marker and the third track shows the 3MeK9H3 marker, a closed chromatin marker. (B) The second track shows the 3MeK9H3 marker, a closed chromatin marker and the third track shows an AcK9H3 marker, an open chromatin marker.

FIG. 3 shows a typical “imprinting signature” for the PLAGL1 gene. PLAGL1 demonstrates a simultaneous active (acetylated K9) and an inactive (tri-methyl K9) at the location of the CpG island of the PLAGL1 promoter.

FIG. 4 shows a typical “imprinting signature” for the N-myc gene. NMYC demonstrates a simultaneous active (acetylated K9) and an inactive (tri-methyl K9) at the location of the CpG island of the NMYC promoter.

FIG. 5 shows a typical “imprinting signature” for the BAT2 gene. BAT2 demonstrates a simultaneous active (acetylated K9) and an inactive (tri-methyl K9) at the location of the CpG island of the BAT2 promoter.

### DETAILED DESCRIPTION OF THE INVENTION

The present invention broadly relates to a method for the rapid identification and monitoring of epigenetic modifications, such as imprinted genes. The method is based on the idea that one copy of an imprinted gene is active on the first chromosome and the other copy is inactive on the second chromosome. The promoter of the active gene is hypothesized to reside in an open chromatin region of the first chromosome and the promoter of the inactive copy of the imprinted gene resides on a condensed chromatin region of the second chromosome. By searching for chromatin structure markers that are indicative of the open and condensed chromatin in the same region, it is possible to rapidly identify the location of imprinted genes in the genome using array based technology, such as ChIP Chip™. Alternatively, loss of imprinting can be identified using other array or PCR based techniques by observing the presence of only open chromatin structure markers in genomic regions where overlapping open and closed chromatin markers are generally observed. Thus, the imprinted genes and the loss of such imprinting, which may be associated with disease can be identified as described herein.

In general, Chromatin Immunoprecipitation (ChIP) is a method widely used to study in vivo protein-DNA interactions (see Solomon, et al. (1988) Cyclin in fission yeast. Cell 54: 738-739; and Orlando, V. (2000) Mapping chromosomal proteins in vivo by formaldehyde crosslinked chromatin immunoprecipitation. Trends Biochem. Sci. 25, 99-104; both are incorporated herein by reference in their entirety). Traditionally, this technique has been used to confirm whether a transcription factor (TF) binds to a particular DNA sequence in vivo. Using this technique living cells are first treated with formaldehyde and then broken apart. The chromosomes are sheared by sonication, and the cross-linked chromatin DNA fragments are immunoprecipitated using a specific antibody against the TF. The enrichment of a particular sequence in the immunoprecipitates is tested by PCR with a pair of gene-specific primers and visualized using gel electrophoresis. Analysis of the PCR product yield compared to a non-immunoprecipitated control determines whether the protein of interest is bound to the DNA region tested. However, each region of DNA must be tested individually by PCR. Thus, unlike the present invention, the ChIP technique is generally limited to a small set of DNA regions that are selected for analysis.

Researchers have found the immunoprecipitates contain all target sequences for a TF. Sequencing the immunoprecipitated DNA fragments has been attempted, to identify factor target genes (Weinmann et al., (2001) The use of chromatin immunoprecipitation to clone novel E2F target promoters. Mol. Cell. Biol. 21: 6820-6832; and Weinmann, et al., (2002) Isolating human transcription factor targets by coupling chromatin immunoprecipitation and CpG island microarray analysis. Genes & Dev. 16: 235-244.) For example, Weinmann et al., cloned the DNA immunoprecipitated with E2F4 antibody from human cells, biochemically verified E2F protein binding of a dozen clones, and sequenced them to identify several novel E2F target genes. However, in the same study, none of the previously known E2F target genes were found, casting doubts on the efficiency of this method. Apparently, it is limited by an inefficient cloning step and the slow, painstaking biochemical process to eliminate false positive clones one by one.

Further, applicants have coupled chromatin immunoprecipitation with Maskless Array Synthesis (MAS) microarray technology to develop the ChIP Chip™ technique, which is available through NimbleGen Systems (Madison, Wis.). The MAS microarray technology is described in U.S. Pat. No. 6,375,903 and U.S. Pat. No. 5,143,854, each of which is herein incorporated by reference in its entirety. The disclosure of U.S. Pat. No. 6,375,903 enables the construction of the maskless array synthesizer (MAS) instruments in which light is used to direct synthesis of the DNA sequences, the light direction being performed using a digital micromirror device (DMD). Using a MAS instrument, the selection of DNA sequences to be constructed in the microarray is under software control so that individually customized arrays can be built to order. In general, MAS based DNA microarray synthesis technology allows for the parallel synthesis of over 800,000 unique oligonucleotides in a very small area of on a standard microscope slide. The microarrays are generally synthesized by using light to direct which oligonucleotides are synthesized at specific locations on an array, these locations are called features.

Using the ChIP Chip™ technology, applicants and other researchers have been able to identify TF targets in yeast cells (Lee et al., Msx1 Cooperates with Histone H1b for Inhibition of Transcription and Myogene, Science (2004) 304: 1675-1678) and human cells (Kim et al., (2005) A high-resolution map of active promoters in the human genome. Nature. 436:876-880.). Instead of sequencing the immunoprecipitated DNA, protocols have been developed to amplify and fluorescently label the DNA, which is then hybridized to DNA microarrays containing probes to intergenic regions of the yeast or human genome. As a control, genomic DNA is labeled using a different fluorescent dye and hybridized to the same array. The probes that show a significantly stronger signal in the IP-enriched DNA channel indicate the corresponding intergenic regions are bound by the TF in vivo and can be plotted as the ratio of IP-enriched/genomic DNA control along the genomic position of the probes.

In accordance with the invention, ChIP Chip™ technology can also be used to study DNA chromatin structure. Specifically, ChIP Chip™ can be used to identify DNA on the basis of enzymatic modifications of the histone proteins which are used in packaging DNA. It is currently thought that modifications to the histone proteins are involved in determining the packing density of DNA. Suitable histone modifications include but are not limited to histone H3 and H4 acetylation, histone H3 methylation, histone H1 phosphorylation. Modifications such as 3 methyl groups on the lysine 9 of histone 3 (3MeK9H3) are thought to be associated with condensed chromatin that is not accessible to the transcription machinery. Other modifications such as an acetyl group on the lysine 9 of histone 3 (AcK9H3) are thought to be associated with open chromatin that is accessible to the transcription machinery. These histone modifications are often found in the promoters of genes and are thought to be a means of regulating gene transcription. The DNA is wrapped tightly around the histones and these interactions are easily captured with crosslinking. ChIP Chip™ has been found to be effective in mapping histone modifications.

As used herein the term “histone” refers to a protein that forms the unit around which DNA is coiled in the nucleosomes of eukaryotic chromosomes. In eukaryotes, genomic DNA is packaged with histone proteins into chromatin, compacting DNA some 10,000-fold. The DNA-histone structure is the nucleosome, typically composed of an octamer of the four core histones H2A, H2B, H3 and H4 and 146 basepairs of DNA wrapped around the histones. Each core histone is composed of a structured domain and an unstructured amino-terminal ‘tail’ of 25-40 residues. This unstructured tail extends through the DNA gyres and into the space surrounding the nucleosomes. The nucleosome is not compatible with gene expression. Re-organization of the nucleosome is required for transcription factors and RNA polymerase to have access to the DNA for transcription.

As used herein the term “histone modification” refers to the post-translational modifications of histones. Post-translational modifications of histone amino-termini have long been thought to play a central role in the control of chromatin structure and function. A large number of covalent modifications of histones have been documented, including acetylation, phosphorylation, methylation, ubiquitination, and ADP ribosylation that take place on the amino terminus “tail” domains of histones. Such diversity in the types of modifications and the remarkable specificity for residues undergoing these modifications suggest a complex hierarchy of order and combinatorial function that still remains unclear. Covalent post-translational modifications of the amino-terminal tails of histones regulate the transcriptional ‘on’ or ‘off’ states of chromatin and influence chromosome condensation and segregation. The three best characterized histone modifications include, covalent modifications: histone acetylation, histone methylation and cytosine methylation.

As used herein, the term “open chromatin” refers to a region of chromatin that is at least 10-fold more sensitive to the action of an endonucleoase, e.g., DNAse I, than surrounding regions. Because opening of the chromatin is a prerequisite to transcription activity, DNAse I sensitivity provides a measure of the transcriptional potentiation of a chromatin region; greater DNAse sensitivity generally corresponds to greater transcription activity. DNAse hypersensitivity assays are described by Weintraub & Groudine, 1976, Science 193: 848-856, incorporated herein by reference. “Highly transcribed” or “highly expressed” regions or genes are regions of open chromatin structure that are transcribed. Recently researchers have found that regions that are rich in genes tend to be in open chromatin structures, whereas regions poor in genes tend to be in compact chromatin. However, open chromatin can contain inactive genes and compact chromatin can contain active genes. (See Bickmore, et al (2004) Chromatin Architecture of the Human Genome: Gene-Rich Domains Are Enriched in Open Chromatin Fibers, Cell, Vol 118, 555-566, 3.)

The present invention encompasses a variety of related embodiments involving methods for identifying and monitoring epigenetic modifications, such as gene imprinting and the loss of gene imprinting through the methods described below. As used herein an epigenetic change refers to modifications in gene expression that are controlled by heritable but potentially reversible changes in DNA methylation and/or chromatin structure. DNA methylation is a post-replication process by which cytosine residues in CpG sequences are methylated, forming gene-specific methylation patterns. Housekeeping genes possess CpG-rich islands at the promoter region that are unmethylated in all cell types, whereas tissue-specific genes are methylated in all tissues except the tissue where the gene is expressed. These methylation patterns obviously correlate with gene expression. Furthermore, genes that do not obey Mendel's genetic rules, being expressed monoallelically in a parent-of-origin fashion, such as imprinted genes, are imprinted by an epigenetic mechanism.

In order to efficiently enable the identification of imprinted genes in a specific tissue or a cell type, applicants developed a method of establishing a genome-wide chromatin structure or imprinting map. In general, the term “imprinting map” illustrates the chromosomal regions of the genome subject to imprinting. Chromosomes that are likely to show imprinting include 2, 6, 7, 11, 14, 15, 16, 20 and X (see Ledbetter D. H. and Engel E. (1995) Hum. Mol. Genet. 4: 1757; Morison I. M. and Reeve A. E. (1998) Hum. Mol. Genet. 7: 2599). Chromosome regions could be labeled according to the phenotype.

Accordingly, in one embodiment, the invention provides for a human chromosomal imprinting map which can have important clinical implications. The basic method includes isolating a genomic DNA sample from a subject. The subject's DNA sample is then analyzed using the applicant's chromatin immunoprecipitation microarray technology referred to as ChIP Chip™. A map of the subject's chromatin structure is generated enabling the identification of chromosomal regions on the map having at least one imprinted gene. As described herein, the imprinted genes are characterized by the presence of at least one set of overlapping closed and open chromatin markers.

In a preferred embodiment, applicants have found the inventive methods work best when three specific criteria are present: 1) open chromatin mark; 2) closed chromatin mark; and 3) these marks occur on an CpG island. CpG islands are regions of unusually high CG content and are usually under positive selection. Specifically, researchers have defined a CpG island as an about 200 to an about 500-bp stretch of DNA with a C+G content of 50% and an observed CpG/expected CpG ratio in excess of about 0.5 or about 0.6. (See Gardiner-Garden, M. and Frommer, M. (1987) J. Mol. Biol. 196, 261-282). Further, detection of regions of genomic sequences that are rich in the CpG pattern is important because such regions are resistant to methylation and tend to be associated with genes which are frequently switched on. The regions rich in the CpG pattern are known as CpG islands.

It is envisioned that the imprinting map according to the invention may be established for different tissues and at different developmental stages. Such an imprinting map can be used to search for a variety of diseases attributed to abnormal or loss of imprinting (characterized by the loss of the closed chromatin marker resulting in the activation of the second allele of a gene) relative to a healthy subject's imprinting map. It is noted that an imprinting map can be prepared for humans as well as other organisms including, but not limited to vertebrates and mammals such as a dog, cat, rabbit, cow, bird, rat, horse, pig, or monkey.

In a related embodiment, the invention provides a method of identifying at least one imprinted gene. The method includes isolating a biological sample from a subject and analyzing chromatin structure of a subject's genome by ChIP Chip™ to establish a map of the subject's chromatin structure. Subsequently, a genomic region is identified on the map having at least one imprinted gene, characterized by the presence of at least one set of overlapping open and closed chromatin markers.

In practicing the invention, the applicants have suitably identified 4 markers for chromatin structure that are indicators for the identification and monitoring of imprinted genes. These four markers include histone modifications such as: 1) acetylated lysine 9 of histone 3 (AcK9H3) an open chromatin marker; 2) tri-methyl lysine 9 of histone 3 (3MeK9H3), a condensed chromatin marker; 3) di-methyl lysine 9 of histone 3 (2MeK9H3), believed to be a temporarily condensed chromatin marker; and 4) RNA polymerase II (Pol II), a protein complex involved in RNA transcription and most likely to be found bound to the open chromatin. Specifically, in a preferred embodiment, the invention uses ChIP Chip™ to map the chromatin structure of a human's genome and to identify genomic regions having overlapping open (AcK9H3) and closed (3MeK9H3) chromatin markers indicating the presence of at least one imprinted gene.

Thus, another embodiment of the invention provides markers of chromatin structure for use in identifying imprinted genes and the loss of such gene imprinting. The preferred markers for identifying gene imprinting include histone modifications such as: 1) acetylated lysine 9 of histone 3 (AcK9H3); 2) tri-methyl lysine 9 of histone 3 (3MeK9H3); 3) di-methyl lysine 9 of histone 3 (2MeK9H3); and 4) RNA polymerase II (Pol II). Alternatively, the preferred markers for identifying loss of imprinting include only open chromatin markers such as AcK9H3 and Pol II. However, a skilled person will readily appreciate that other open and condensed chromatin markers capable identifying imprinted genes and loss thereof would also be applicable.

In another embodiment, the present invention provides a novel method for identifying a loss of gene imprinting (LOI) in a subject. Generally, methods for detecting loss of imprinting have been quantitative methods for analyzing imprinting status. For example, using quantitative PCR assays, applicants determined that a frequent loss of imprinting in one allele of the IGF2 gene was associated with colon cancer. Thus, the identification of abnormalities in the imprinting of a gene or a population of genes could facilitate diagnosis of a disease or determine a predisposition for a disease (see, for example, U.S. Pat. No. 6,235,474).

The presence or absence of LOI may be detected by examining any condition, state, or phenomenon which causes LOI or is the result of LOI. Causes of LOI, include the state or condition of the cellular machinery for DNA methylation, the state of the imprinting control regions, the presence of trans-acting modifiers of imprinting, the degree or presence of histone deacetylation. State of the genomic DNA associated with the genes or gene for which LOI is being assessed, include the degree of DNA methylation. Effects of LOI, can include the following: relative transcription of the two alleles of the genes or gene for which LOI is being assessed; post-transcriptional effects associated with the differential expression of the two alleles of the genes or gene for which LOI is being assessed; relative translation of the two alleles of the genes or gene for which LOI is being assessed; and post-translational effects associated with the differential expression of the two alleles of the genes or gene for which LOI is being assessed.

Other downstream effects of LOI, include altered gene expression measured at the RNA level, at the splicing level, or at the protein level or post-translational level (i.e., measure one or more of these properties of an imprinted gene's manifestation into various macromolecules); changes in function that could involve, for example, cell cycle, signal transduction, ion channels, membrane potential, cell division, or others (i.e., measure the biological consequences of a specific imprinted gene being normally or not normally imprinted (for example, QT interval of the heart). Another group of macromolecular changes include processes associated with LOI such as histone acetylation, histone deacetylation, or RNA splicing.

In another embodiment, the present invention uses the ChIP Chip™ technology to facilitate the detection of a loss of imprinting. This includes obtaining a biological sample from the subject, the normal biological sample having normal gene imprinting characterized by the presence of at least one set of overlapping closed and open chromatin markers. The biological sample is then screened for loss of imprinting in at least one gene characterized by the presence of at least one closed chromatin marker, such as 3MeK9H3 in a region known to have normal gene imprinting. This method may be used as a diagnostic test or assay to determine a predisposition to a disease associated with loss of imprinting, such as cancer.

It is envisioned that a diagnostic assay to determine a predisposition to a disease associated with loss of imprinting could use virtually any biological sample containing preferably genomic DNA (other than pure red blood cells) is suitable. For example, convenient tissue samples include whole blood, saliva, buccal, tears, semen, urine, sweat, fecal material, skin and hair. Thus, the diagnostic assay of genomic imprinting described here using the ChIP Chip™ technology is of considerable practical importance, as this assay does not require tumor tissue. The approach described here also represents the first genetic test that can ascertain a substantial fraction of patients in the general population with cancer or at risk of cancer.

In a related embodiment, the present invention provides a method for detecting the presence of a disease in a subject. The method includes obtaining a biological sample from the subject, the normal biological sample having normal gene imprinting characterized by the presence of at least one set of overlapping closed and open chromatin markers. The biological sample may include cells from blood or tissue and preferably includes genetic information. The sample is then screened for loss of imprinting in at least one gene characterized by searching for the loss of the closed chromatin marker in a region known to have normal gene imprinting, wherein loss of imprinting (LOI) indicates presence of the disease.

In general, the presence or absence of LOI may be determined for any gene or genes which are known to normally exhibit imprinting. Currently there are about 22 genes which are known to be normally imprinted (see Feinberg in The Genetic Basis of Human Cancer, B Vogelstein & K Kinzler, Eds., McGraw Hill, 1997, which is incorporated herein by reference). Examples of such genes include, but are not limited to, IGF2, H19, p57/KIP2, KvLQT1, TSSC3, TSSCS, and ASCL2. However, it is expected that additional genes which normally exhibit imprinting will be discovered and the LOI of such genes may be the target of the present methods and are therefore encompassed in the present embodiments.

It is envisioned that such methods would be useful in diagnosing diseases such as cancer, birth defects, mental retardation, obesity, neurological disease, diabetes, or gestational diabetes, among others. Specifically, with respect to cancer, it is envisioned that the method would be particularly useful in detecting colorectal cancer, esophageal cancer, stomach cancer, leukemia/lymphoma, lung cancer, prostate cancer, uterine cancer, breast cancer, skin cancer, endocrine cancer, urinary cancer, pancreatic cancer, other gastrointestinal cancer, ovarian cancer, cervical cancer, head cancer, neck cancer, or adenomas. Also, with respect to the genes being monitored for imprinting or loss of imprinting, they include IGF2, H19, p57/KIP2, KvLQT1, TSSC3, TSSC5, or ASCL2.

In a related embodiment, the invention provides a method for assessing the risk of contracting a disease in a subject. The method includes obtaining a biological sample from the subject, the normal biological sample having normal gene imprinting characterized by the presence of overlapping closed and open chromatin markers. The biological sample is then screened for loss of imprinting in at least one gene characterized by the loss of the closed chromatin marker in a region known to have normal gene imprinting; wherein loss of imprinting indicates a risk for contracting the disease. Other embodiments may include a DNA-based blood test for the general population, described below.

Similarly, the present invention provides methods that identify cancer risk at high frequency in the general population. In a preferred embodiment a positive blood test would confer increased risk of cancer, and potentially may be used to identify high-risk patients in the general population for increased cancer surveillance. The method provides an additional advantage in that a negative test could serve to exclude patients who may have a positive family history from repeat invasive examinations. Furthermore, the test can be performed on biological samples, such as blood.

In one embodiment, it is preferred that a DNA rather than RNA-based test may be used to assess predisposition to disease. Accordingly, in certain embodiments, methods of the present invention include analyzing the genomic DNA for loss of imprinting determined through the presence of a at least one condensed chromatin marker where overlapping open and closed chromatin markers exist in an imprinting map of a general population. It is envisioned that such a test could be administered in a clinic using a diagnostic tool, such as quantitative PCR.

A method according to the present invention can be performed during routine clinical care, for example as part of a general regular checkup, on a subject having no apparent or suspected neoplasm such as cancer. Therefore, the present invention in certain embodiments, provides a screening method for the general population. The methods of the present invention can be performed at a younger age than present cancer screening assays, for example where the method can be performed on a subject under 65.

If the biological sample of the subject is found to exhibit LOI, for example as the result of the loss of closed chromatin markers in genomic regions having generally contained overlapping open and closed markers, then that subject would be identified as having an increased probability of having cancer. In such an embodiment, both alleles of a gene are expressed normally, however, the genomic region is no longer imprinted. In such an embodiment, it may be preferred to perform one or more diagnostic tests to probe for the possibility of cancer being present in the subject. In addition, it may also be preferred to prescribe a schedule for performing additional diagnostic tests in the future, even if no cancer is present at the time LOI is detected. Further diagnostic tests known in the art, such as but not limited to a chest X-ray, carcinoembryonic antigen (CEA) or prostate specific antigen (PSA) level determination, colorectal examination, endoscopic examination, MRI, CAT scanning may be carried out to probe for the possibility of cancer being present in the subject.

In a related embodiment, the present invention provides a novel method for detecting a disease or measuring the predisposition of a subject for developing a disease in the future by obtaining a biological sample from a subject; and screening the biological sample for the presence of abnormal imprinting or loss of imprinting (LOI) characterized by the loss of closed chromatin markers. It is envisioned that screening for LOI can be performed very efficiently in a clinic using quantitative PCR based methods.

The biological sample may include any sample which is conveniently taken from the patient and contains sufficient information to yield reliable results. However, it is possible to obtain samples which contain smaller numbers of cells and then enrich the cells. In addition, with certain highly sensitive assays (e.g., RT-PCR when the gene of interest (e.g., IGF) is abundant, and other methods like DNA methylation even when IGF2 not abundant) it is possible to get sample size down to single cell level. Also, the sample need not contain any intact cells, so long as it contains sufficient biological material (e.g., protein; genetic material, such as DNA or RNA; etc.) to assess the presence or absence of LOI in the subject.

In another embodiment of the invention, a method is encompassed for determining if a preimplantation embryo has an increased risk of developing a disease due to loss of imprinting. The method includes identifying at least one gene that is imprinted in at least one control embryo by mapping chromatin structure of the control embryo genome using ChIP Chip™, wherein the imprinted gene is characterized by the presence of at least one set of overlapping closed and open chromatin markers. A biological sample is then isolated from the preimplantation embryo. The chromatin structure of the preimplantation embryo's genome is mapped using ChIP Chip™. The results may show that a preimplantation embryo has an increased risk of developing a disease due to loss of imprinting status relative to the imprinting status of the control embryo; wherein the loss of imprinting is characterized by the loss of at least one closed chromatin marker. Similarly, it is also envisioned that the ChIP Chip™ technology can be used to identify imprinted genes associated with a disease states, different tissues and/or at different developmental stages.

Although array based methods, such as ChIP Chip™, are the preferred method for identifying imprinted genes, alternative embodiments to screen for LOI in clinics or pre-clinical diagnostic settings are encompassed within the scope of invention. These alternative embodiments, include but are not limited to polymerase chain reaction (PCR) based methods, such as real-time or quantitative PCR (Q-PCR), which are diagnostic tools for quantifying starting amounts of DNA, cDNA, or RNA templates via a fluorescent reporter molecule that increases as PCR product accumulates with each cycle of amplification. This tool has greatly enhanced several areas of research including gene expression analysis and genotyping assays.

Such, diagnostic tools known in the art can be used to quickly assay a small number of genomic regions. A practical advantage of the PCR-based methods is that they are less expensive for performing a small number of diagnostic tests. Additionally, Q-PCR is routinely used to validate array results from ChIP Chip™ experiments.

The following examples are provided as further non-limiting illustrations of particular embodiments of the invention.

### EXAMPLES

### Example 1

ChIP Chip™ for Discovery and Monitoring of Imprinted Genes

In order to determine which region of a genome contains imprinted genes, applicants performed ChIP Chip™ experiments to identify regions having overlapping open and closed chromatin markers, by virtue of their association with preferably, modified histone markers such as AcK9H3; 3MeK9H3; 2MeK9H3; and RNA polymerase II.

Briefly, to perform ChIP-chip™, cells were treated chemically to cross-link DNA binding proteins to their binding sites. DNA from these cells was isolated, fragmented and enriched by immunoprecipitation with antibodies directed to the modified histones and RNA polymerase of interest (see generally, Ng et al., Genes Dev. 16, 806-819 (2002); Wyrick et al., Science 290, 2306-2309 (2002); Weinmann, et al., Genes Dev. 16, 235-244 (2002)).

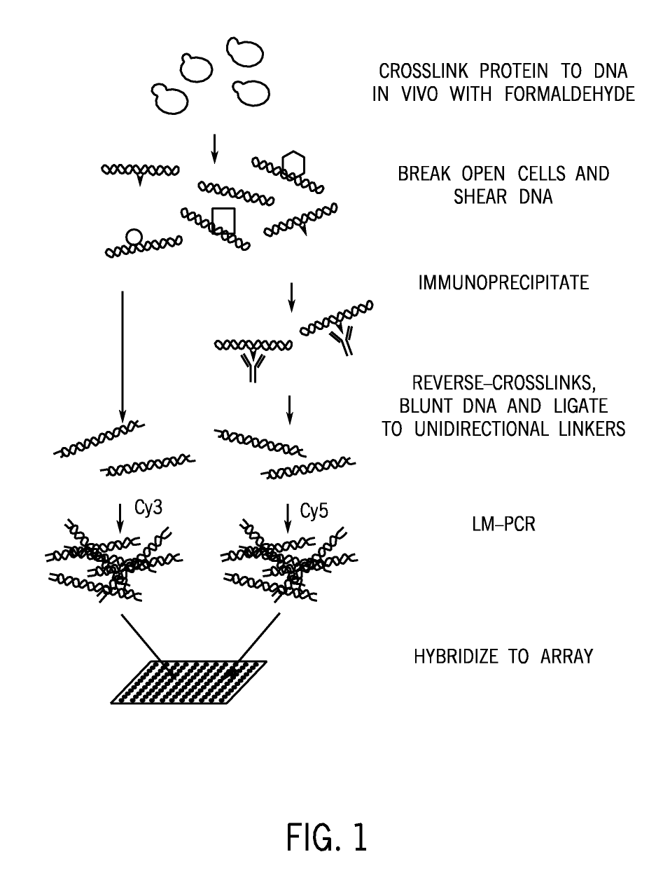

This enriched population was then amplified by LM-PCR. As depicted in FIG. 1, the present method is not limited to amplifying individual DNA regions by performing PCR with specific primers. Rather the entire genome is amplified using a Ligation-Mediated PCR (LM-PCR) strategy well known in the art. The amplified DNA may be fluorescently labeled by including fluorescently-tagged nucleotides in the LM-PCR reaction. It may be labeled using a random prime method that uses labeled primers or incorporates fluorescent probes during the random priming reaction. The labeled LM-PCR amplified DNA population is then hybridized to DNA microarrays (ChIP Chip™) in parallel with controls where the antibody has been omitted. The DNA microarray contains features representing all or a subset (e.g., a chromosome or chromosomes) of the genome. The fluorescent intensity of each feature on the microarray relative to a non-immunoprecipitated control demonstrates whether the protein of interest bound to the DNA region located on the feature.

Applicants note that the ability of the MAS microarray technology to synthesize long (50-60-mer) oligo, high-density arrays assures sufficient signal-to-noise ratio to distinguish true positives from background and assures the capacity to map large regions of the genome of interest. The probe capacity of these arrays also allows up to 40 MB of researcher-specified sequence to be surveyed for high-resolution open and closed chromatin markers. Hence, the methods described herein allow the detection of protein-DNA interactions (overlapping open and closed chromatin) across the entire genome. This genome-wide location analysis method described herein allows imprinted genes to be monitored across an entire genome.

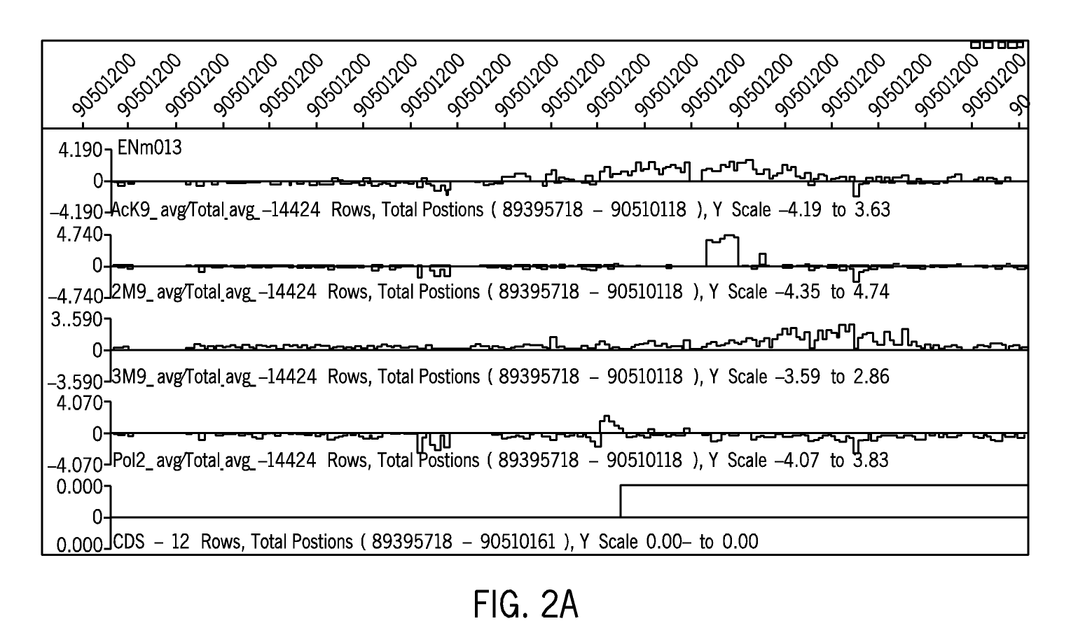

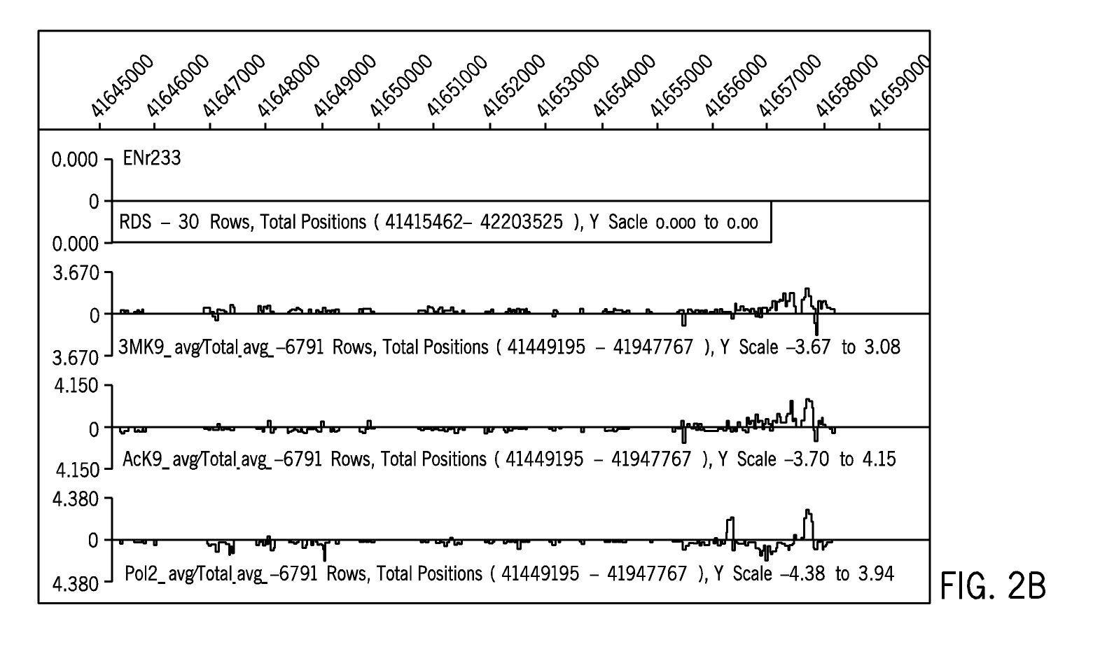

The results provided in FIGS. 2A and B show plots that contain overlapping regions with both open (AcK9H3) and closed (3MeK9H3) chromatin markers. The data indicate that chromatin immunoprecipitation using antibodies to modified histone markers can be used to identify imprinted regions in the genome. The results of this and other related experiments demonstrate the potential of ChIP-chip™ microarray analysis to greatly enhance our understanding of transcription regulation. More specifically, the invention enables genome-wide identification of imprinted genes, which can serve as a diagnostic screen for the loss of imprinting which can indicate the presence of disease and/or the risk of developing a disease.

### Example 2

Microarray Analysis of the Co-occurrence of Silenced and Active Chromatin Marks as a High Throughput Method for Identifying Novel Imprinted Genes

Applicants conducted a series of microarray experiments to efficiently screen for or identify coincident “active” and “silent” chromatin marks, with or without the presence of a CpG island or GC-rich sequence, regardless of the methylation state of that CpG island in the particular cell type.

Chromatin Immunoprecipitation

Chromatin Immunoprecipitation (ChIP) was performed on cultured HeLA cells and 293 cells utilizing the well known ChIP protocol from the Farnham lab published electronically and publicly available on Dec. 12, 2005 at http://genomecenter.ucdavis.edu/farnham/farnham/protocols/chips.html, combined with a ligation mediated PCR technique (LMPCR) based on the method of Ren et al. (2000) Science. 290, pg. 2306 (both of which are incorporated by reference in their entirety).

The materials to conduct the ChIP analysis were commercially available. The Histone H3 (tri methyl K9) antibody (cat.# ab8898) and the CTCF antibody (cat.# ab10571) were obtained from Abcam Inc. (Cambridge, Mass.), and the Histone H3 (anti-acetyl K9, cat.# 07-352) was obtained from Upstate Biotechnology.

Microarray Design

A 50-mer tiled microarray was designed based on the NimbleGen maskless array synthesis technology as described herein above. This microarray encompassed a majority of the known imprinted genes (80%), according to The Catalogue of Imprinted Genes, incorporated by reference herein (see Morison I M, Paton C J, Cleverley S D. The imprinted gene and parent-of-origin effect database. Nucleic Acids Research 2001; 29(1): 275-276 or Morison I M, Ramsay J P, Spencer H G. A census of mammalian imprinting. Trends in Genetics 2005 and publicly available database at URL: www.otago.ac.nz/IGC). The microarray also encompassed some regions thought to harbor unknown imprinted genes (chromosomes 6, 7, 11, 13, 14, 16, 18 and 22).

Data Analysis

Data were analyzed using the SignalMap™ program developed by NimbleGen Systems (Madison, Wis.). SignalMap™ is a software tool that enables visualization of NimbleGen array data.

Results

Microarray analysis of the chromosomal regions provided validation that the inventive method is effective for identifying and monitoring epigenetic changes, such as imprinted genes, through the co-occurrence of silenced and active chromatin marks. It is noted that a significant fraction of imprinted genes showed a co-occurrence of silenced and active chromatin on top of a CpG island (triple sign). For example, FIG. 1 shows that the PLAGL1 gene has a typical triple sign “imprinting signature” indicating simultaneous active (acetylated K9) and an inactive (tri-methyl K9) at the location of the CpG island of the PLAGL1 promoter.

In the first cell line (HeLa cervical carcinoma) examined, 60 Mb of the genome was screened (2%) and 5 of the 43 imprinted genes showed this triple sign. This is an enrichment of over 25-fold, using a single threshold and a single cell line. This number would be expected to increase substantially using alternative thresholds and additional cell lines. This is true given the tissue specificity of imprinted genes, so one would not expect to find all imprinted genes in a single cell type. There is currently no workable method for genome-scale imprinted gene discovery. Therefore, the claimed method would be very useful for efficiently identifying and monitoring epigenetic modifications.

The method also identifies genes that have an epigenetic mark associated with disease, even if the gene is not imprinted. There is no alternative to this method for identifying such genes a priori, since mouse embryo experiments, which are costly and cumbersome would not work at all. An example of such a gene is N-myc (FIG. 2), which also showed a striking triple sign, an imprinting signature. NMYC demonstrates a simultaneous active (acetylated K9) and an inactive (tri-methyl K9) at the location of the CpG island of the NMYC promoter. It should be noted that the Signal Map™ screen can easily be extended to the entire genome (50× greater coverage) by increasing the number of chips.

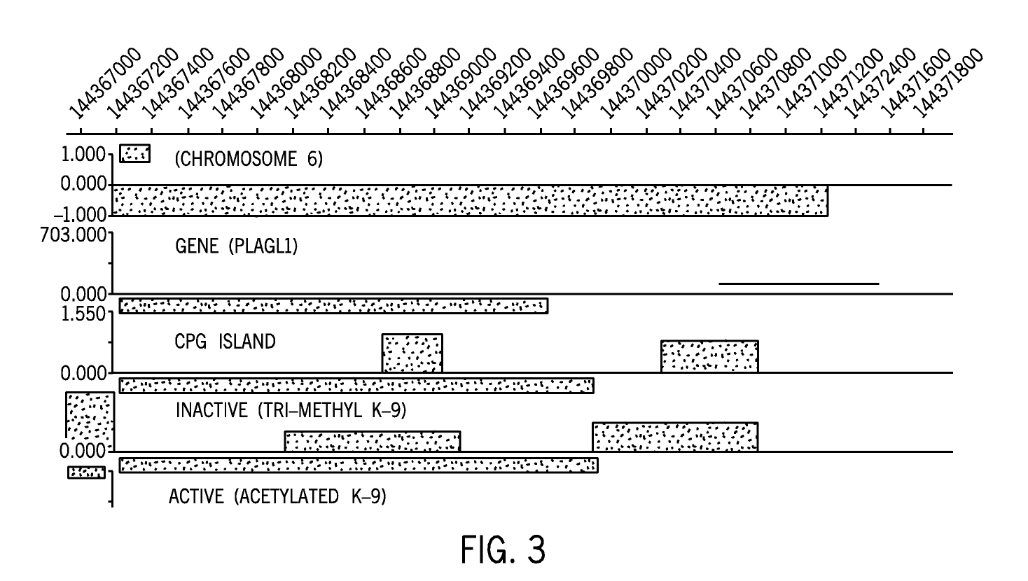

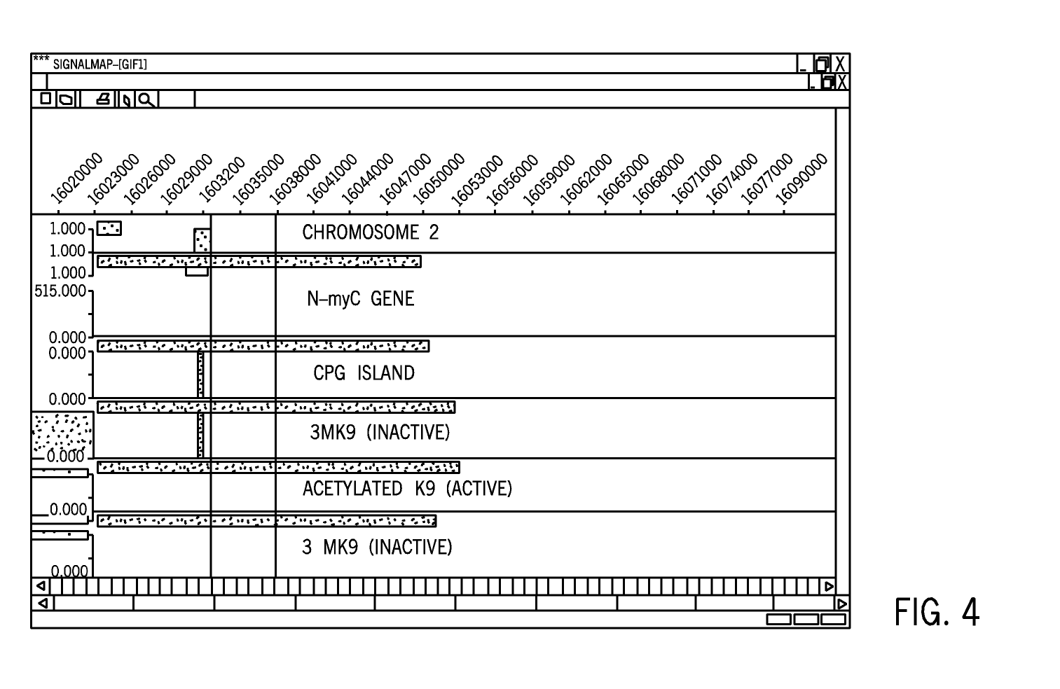

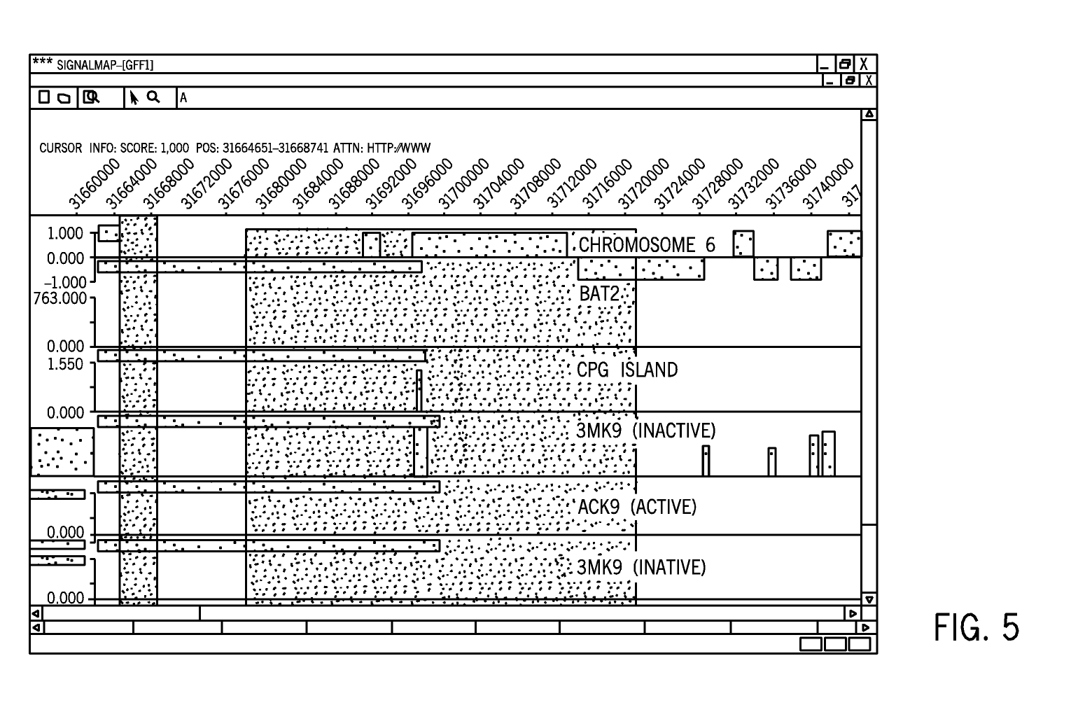

Applicants also identified 4 other genes within this 60 Mb region that also demonstrated this triple sign feature, but are not known to be imprinted (see Table 1 below). One of the genes identified is BAT2 (HLA-B associated transcript 2). FIG. 3 shows BAT2 having a typical “imprinting signature” indicating that BAT2 is simultaneously active (acetylated K9) and inactive (tri-methyl K9) at the location of the CpG island of the BAT2 promoter.

TABLE 1

| Genes not known to be imprinted that demonstrated | NCBI |
|---|---|
| triple sign in HeLa cells: | accession no.: |
| Homo sapiens HLA-B associated transcript 2 (BAT2) | BC060668 |
| Homo sapiens chromosome 6 open reading frame 106 | NM_024294 |
| (C6orf106) | |
| Homo sapiens forkhead box F1 (FOXF1) | NM_001451 |
| Homo sapiens forkhead box C2 (MFH-1, mesenchyme | NM_005251 |
| forkhead 1) (FOXC2) | |

It is also envisioned that the invention may be used to classify genes within a cell type as active or inactive. Specifically, applicants have compiled supplementary tables listing all genes that had the co-occurrence of an active signal and a CpG island (active genes) as well as data on genes that had a co-occurrence of an inactive signal and a CpG island (inactive genes).

In summary, applicants have demonstrated that analyzing “active” and “silenced” chromatin through a high-throughput microarray approach can be utilized to find novel imprinted genes. Because of the tissue-specific nature of imprinting it is necessary to have a high throughput method that can be used on a multitude of tissues. Accordingly, using this invention it should be possible to tile through any genome and identify at least one imprinted gene to determine if an individual has a predisposition to a particular disease.

Each piece of literature cited herein is incorporated entirely herein by reference.

It is understood that certain adaptations of the invention described in this disclosure are a matter of routine optimization for those skilled in the art, and can be implemented without departing from the spirit of the invention, or the scope of the appended claims.

We claim:

1. A method of identifying at least one imprinted gene, the method comprising the steps of:
(a) isolating a biological sample from a subject;
(b) analyzing chromatin structure of the subject's genome to establish a map of the subject's chromatin structure; and
(c) identifying a genomic region on the map having at least one imprinted gene, characterized by the presence of at least one set of overlapping open and closed chromatin markers.

2. The method of claim 1 wherein the subject is human.

3. The method of claim 1 wherein the markers for chromatin structure comprise modified histones or RNA polymerase II.

4. The method of claim 3 wherein the modified histones are selected from the group consisting of acetylated lysine 9 of histone 3 (AcK9H3), tri-methyl lysine 9 of histone 3 (3MeK9H3), and di-methyl lysine 9 of histone 3 (2MeK9H3).

5. The method of claim 1 wherein the analyzing step is performed by chromatin immunoprecipitation microarray, ChIP Chip™.

6. A chromatin structure marker used for identifying imprinted genes in a eukaryotic genome.

7. A chromatin structure marker used for identifying imprinted genes in a eukaryotic genome, wherein the marker comprises modified histones or RNA polymerase II.

8. A method for detecting a loss of gene imprinting in a subject, the method comprising the step of:
(a) obtaining a biological sample from the subject, the normal biological sample having normal gene imprinting characterized by the presence of at least one set of overlapping closed and open chromatin markers;
(b) screening the biological sample for loss of imprinting in at least one gene; and
(c) detecting the loss of gene imprinting in at least one gene, wherein the gene exhibiting the loss of imprinting is characterized by the loss of at least one closed chromatin marker relative to normal gene imprinting.

9. The method of claim 8 wherein the closed chromatin marker is 3MeK9H3.

10. Use of the method of claim 8 to diagnose a predisposition to a disease associated with loss of imprinting, such as cancer.

11. The method of claim 8 wherein the screening step is performed using ChIP Chip™ or quantitative PCR.

12. A method for detecting the presence of a disease in a subject, the method comprising the step of:
(a) obtaining a biological sample from the subject, the normal biological sample having normal gene imprinting characterized by the presence of at least one set of overlapping closed and open chromatin markers;
(b) screening the biological sample for a loss of imprinting in at least one gene, wherein the gene exhibiting the loss of imprinting is characterized by the loss of at least one closed chromatin marker relative to normal gene imprinting; and
(c) detecting the loss of imprinting in at least one gene, such that the loss of imprinting is indicative of the presence of a disease.

13. The method of claim 12 wherein the screening can be done by array based methods, such as ChIP Chip™ or quantitative PCR.

14. The method of claim 12, wherein the disease is selected from the group consisting of cancer, birth defects, mental retardation, obesity, neurological disease, diabetes, and gestational diabetes.

15. The method of claim 14, wherein the cancer is selected from the group consisting of colorectal cancer, esophageal cancer, stomach cancer, leukemia/lymphoma, lung cancer, prostate cancer, uterine cancer, breast cancer, skin cancer, endocrine cancer, urinary cancer, pancreatic cancer, other gastrointestinal cancer, ovarian cancer, cervical cancer, head cancer, neck cancer, and adenomas.

16. The method of claim 12, wherein the at least one gene is selected from the group consisting of IGF2, H19, p57/KIP2, KvLQT1, TSSC3, TSSC5, or ASCL2.

17. The method of claim 12, wherein the biological sample are cells selected from the group consisting of blood, saliva, buccal, tears, semen, urine, sweat, fecal material, skin and hair.

18. A method for assessing a risk of contracting a disease in a subject, the method comprising the step of:
(a) obtaining a biological sample from the subject, the normal biological sample having normal gene imprinting characterized by the presence of overlapping closed and open chromatin markers;
(b) screening the biological sample for a loss of imprinting in at least one gene, wherein the gene exhibiting the loss of imprinting is characterized by the loss of at least one closed chromatin marker relative to normal gene imprinting; and
(c) detecting the loss of imprinting in at least one gene, wherein the loss of imprinting is indicative of the risk for contracting the disease.

19. The method of claim 18 wherein the loss of imprinting is characterized by the absence of overlapping closed and open chromatin markers; relative to the imprinting status of the healthy individual.

20. A method for determining if a preimplantation embryo has an increased risk of developing a disease due to loss of imprinting, the method comprising the steps of:
(a) identifying at least one gene that is imprinted in at least one control embryo by mapping chromatin structure of the control embryo genome, wherein the imprinted gene is characterized by the presence of at least one set of overlapping closed and open chromatin markers;
(b) isolating a biological sample from the preimplantation embryo;
(c) mapping chromatin structure of the preimplantation embryo's genome; and
(d) identifying that the preimplantation embryo has an increased risk of developing a disease due to loss of imprinting status in the preimplantation embryo relative to the imprinting status of the control embryo; wherein the loss of imprinting is characterized by the loss of at least one closed chromatin marker.

21. The method of claim 20 wherein the loss of imprinting is characterized by the absence of overlapping closed and open chromatin markers; relative to the imprinting status of the embryo.

22. A method of establishing an imprinting map, the method comprising the steps of:
(a) isolating a genomic DNA sample from a subject;
(b) analyzing the subject's genome; and
(c) generating a map of the subject's chromatin structure; and
(d) identifying chromosomal regions on the map having at least one imprinted gene characterized by the presence of at least one set of overlapping closed and open chromatin markers, such that an imprinting map is established.

23. The method of claim 22 wherein the imprinting map is established in different tissues.

24. The method of claim 22 wherein the imprinting map is established at different developmental stages.

25. The method of claim 22 wherein the imprinting map is used to search for disease.

26. The method of claim 22 wherein the analyzing step is done using array based technology, such as, ChIP Chip™.
